(19) United States
(12) Patent Application Publication
(10) Pub. No.: US 2008/0269780 A1
(43) Pub. Date: Oct. 30, 2008

(54) Epilation Head and Epilation Device

(75) Inventors: Pedro Sanchez-Martinez, Kronberg (DE); Robert Damaschke, Eschborn (DE)

Correspondence Address: FISH & RICHARDSON PC, P.O. BOX 1022, MINNEAPOLIS, MN, 55440-1022, US

(73) Assignee: BRAUN GMBH, Kronberg (DE)

(21) Appl. No.: 11/576,108

(22) PCT Filed: Jul. 29, 2005
(86) PCT No.: PCT/EP05/08231

§ 371 Date: Apr. 13, 2007

(30) Foreign Application Priority Data
Oct. 1, 2004 (DE) 10 2004 047 873.2

(51) Int. Cl. A61B 17/50

(52) U.S. Cl. 606/133

(57) ABSTRACT

A motor-driven epilation head, and epilation apparatus for use therewith, The epilation head has a rotatable cylinder operable to turn about an axis of rotation. The rotatable cylinder includes clamping devices operable to take hold of and pluck the hairs and actuating elements operable for actuating the clamping devices. The rotatable cylinder has at least one pressure element with a control surface that is at least intermittently contacted by the actuating elements in a sliding fashion. The actuating elements as well as the pressure element are made of a plastic material.

### TECHNICAL FIELD

The invention pertains to a motor-driven epilators and associated epilation heads and clamps.

### BACKGROUND

Epilation apparatuses serve for removing hairs, if possible inclusive of their roots. Certain epilation apparatuses are designed, for example, such that the hairs are clamped by means of clamping devices and plucked due to a movement of the clamping devices relative to the skin. Such clamping devices need to be closed in a predetermined position in order to take hold of the hairs. Then, the clamping devices need to be displaced into another predetermined position in the closed state together with the clamped hairs and ultimately opened again in order to release the plucked hairs. This motion sequence can be realized, for example, by arranging the clamping elements on a rotatable cylinder that is set in rotation by means of an electric motor. The clamping devices can be opened and closed by means of actuating elements that are moved back and forth in the axial direction of the rotatable cylinder and that act upon the clamping devices during these axial movements.

The axial movements of the actuating elements are derived from the rotational movement of the rotatable cylinder. For this purpose, one respective roller is arranged adjacent to the axial end faces of the rotatable cylinder, wherein the actuating elements are guided past these rollers due to the rotational movement of the rotatable cylinder and axially displaced during this process. Since the rollers are supported in a rotatable fashion, hardly any friction occurs during the mechanical contact with the actuating element such that the wear can be maintained at a minimum. However, there is little flexibility in realizing the axial movements of the actuating elements, and therefore the opening and closing movements of the clamping devices. In addition, the space requirement of the rollers is relatively high.

As an example, WO 98/05234 A1 discloses such an epilation apparatus, which additionally features one respective cam plate with a radial cam that extends in the circumferential direction adjacent to the two axial end faces of the rotatable cylinder, such that the actuating elements adjoin the radial cams in a sliding fashion. The radial cams are interrupted in the region of the rollers. Alternatively, radial cams can be utilized that are closed in the circumferential direction. The rollers are not used in this case.

U.S. Pat. No. 5,196,021 discloses an epilation apparatus that features a rotatable cylinder with a multitude of clamping devices that are respectively formed by two movable clamping elements. The clamping elements feature axial projections that respectively extend up to one axial end of the rotatable cylinder and are actuated by a cam element at this location.

JP 2823316 B2 discloses an epilation apparatus that features a rotatable cylinder with clamping devices that are respectively composed of a stationary and a movable clamping element. The closing movement of the clamping devices is realized with rods that act upon the movable clamping elements. For this purpose, the rods are axially displaced by means of cam plates arranged adjacent to the axial end faces of the rotatable cylinder. Spring elements are arranged on the movable clamping elements in order to open the clamping devices.

### SUMMARY

One aspect of the invention features an epilation head for an epilation apparatus for plucking hairs out of human skin. The epilation head is motor-driven and features a rotatable cylinder that can be set in rotation about an axis of rotation. The rotatable cylinder features clamping devices for taking hold of and plucking the hairs, actuating elements for actuating the clamping devices and at least one pressure element with a control surface that is at least intermittently contacted by the actuating elements in a sliding fashion. The actuating elements as well as the pressure element are made of a plastic material.

Numerous options for realizing the control of the clamping devices of an epilation apparatus are offered, such that complex control sequences can be realized. The execution of the closing movement can be exactly predetermined with the shape of the control surface such that an optimal behavior with respect to the closing time, the running noises, etc. can be achieved. It is possible to close each clamping device several times per revolution of the rotatable cylinder. Several actuating elements can be actuated simultaneously with the same force. Additionally, the closing force of the clamping devices can be varied in dependence on the angle of rotation of the rotatable cylinder. This can be used, for example, for transporting plucked hairs into a collection reservoir. Advantageously, in some embodiments, the pressure element only requires very little structural space in the axial direction of the rotatable cylinder. It is also advantageous that a simple and cost-efficient manufacture can be achieved due to the utilization of plastic parts for the actuating elements and the pressure element. The properties of the respective plastic material used make it possible to influence the running noise of the epilation head.

In some embodiments of the epilation head, the control surface of the pressure element and/or the regions of the actuating elements that contact the control surface are supplied with a lubricant. This substantially reduces the wear such that a long service life can be achieved despite the utilization of plastic parts. In other embodiments, the lubricant is stored in a lateral part of the epilation head, in which the pressure element is also arranged. The lubricant used preferably consists of a solid lubricant such that the epilation head can be cleaned with water and tensides.

In some embodiments, it is advantageous that the control surface of the pressure element and/or the regions of the actuating elements that contact the control surface have a surface structure that simplifies the adhesion of lubricants. This makes it possible to achieve a very good lubricating effect and therefore very little wear. For example, the control surface of the pressure element and/or the regions of the actuating elements that contact the control surface may have an open-pored surface. The lubricant adheres particularly well to these surfaces such that a lubricating film of sufficient thickness can be easily formed. The control surface of the pressure element and/or the regions of the actuating elements that contact the control surface may also have a surface that features at least one recess. Lubricant is deposited in the recess and therefore stored directly where it is needed. The control surface of the pressure element may feature a groove that extends in the circumferential direction. In some embodiments, the pressure element and/or the actuating elements are at least partially made of a plastic material, into which a lubricant is incorporated. If no additional lubricant is stored, this provides a high resistance to the influence of cleaning agents such that the lubricant practically causes no soiling of the epilation head at all. If additional lubricant is provided, the emergency running properties, in case of a loss of the additional lubricant, are significantly improved.

In order to realize largely continuous motion sequences, the control surface of the pressure element may be regionally realized in the form of a ramp. In some embodiments, the actuating elements are designed such that their cross section respectively increases toward the region that contacts the control surface of the pressure element. This results in a larger contact surface and therefore reduced surface pressure. This also simplifies the formation of a lubricating film and reduces the wear. The actuating elements are realized, for example, in the form of rods.

In some embodiments, the clamping devices respectively feature a clamping element that is movable relative to the rotatable cylinder, and a clamping element that is stationarily arranged on the rotatable cylinder. This simplifies the assembly in comparison with clamping devices that feature two movable clamping elements. The movable clamping elements are preferably realized in the form of individual components. This is advantageous, for example, with respect to the required structural space. In other embodiments, advantages with respect to the assembly and the required structural space can be achieved by realizing several stationary clamping elements in the form of one integral component. The movable clamping elements are made, for example, of metal and the stationary clamping elements are made, for example, of a plastic material. Spring elements may be provided in order to open the clamping devices. This makes it possible to realize the opening mechanism in a relatively simple fashion and to open the clamping devices very quickly.

Another aspect of the invention pertains to an epilation apparatus for plucking hairs out of human skin that includes a hand-held housing and an epilation head as disclosed herein.

Other aspects, features, and advantages will be apparent from the following detailed description , the drawings, and the claims.

### DESCRIPTION OF DRAWINGS

The figures show:

FIG. 1 is a side view of one embodiment of an epilation head;

FIG. 2 is a sectional representation of the epilation apparatuses in the region of the epilation head;

FIG. 3 is a perspective exploded view of the rotatable cylinder;

FIG. 4 is a perspective representation of an arrangement consisting of a lateral part and a few components of the rotatable cylinder; and

FIG. 5 is a perspective representation of the arrangement according to FIG. 4.

Like reference symbols in the various drawings indicate like elements.

### DETAILED DESCRIPTION

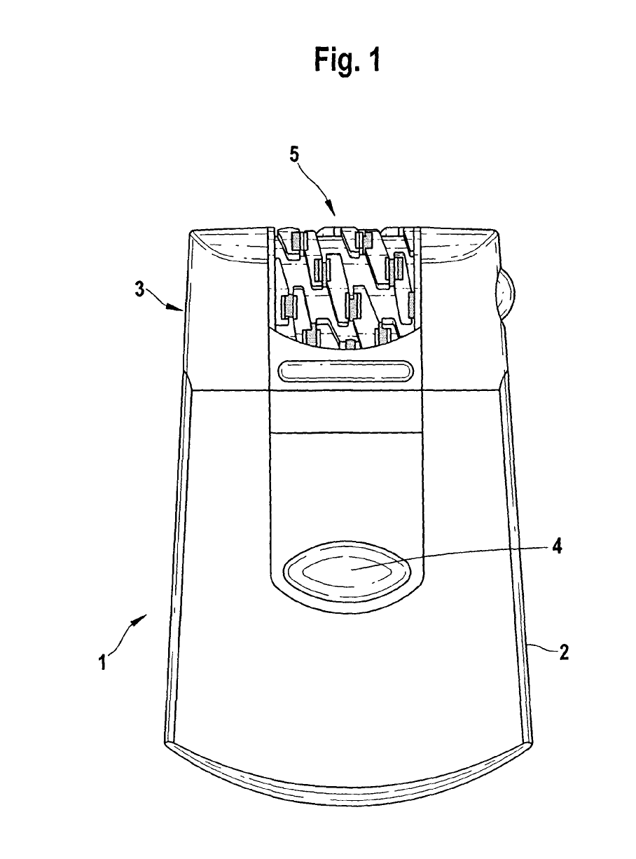

FIG. 1 shows an embodiment of an epilation apparatus 1 in the form of a side view. The epilation apparatus 1 features a housing 2 and an epilation head 3 that is detachably fixed on the housing 2. The housing 2 is shaped such that it can be comfortably held in one hand. A switch 4 for switching the epilation apparatus 1 on and off is arranged on the housing 2. A rotatable cylinder 5 is rotatably suspended in the epilation head 3.

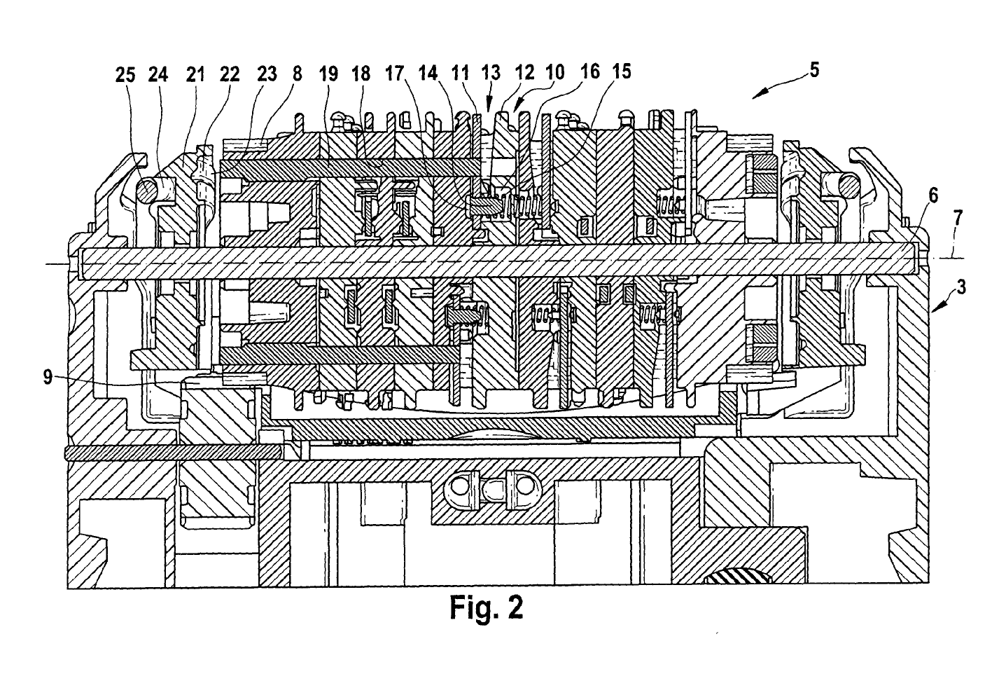

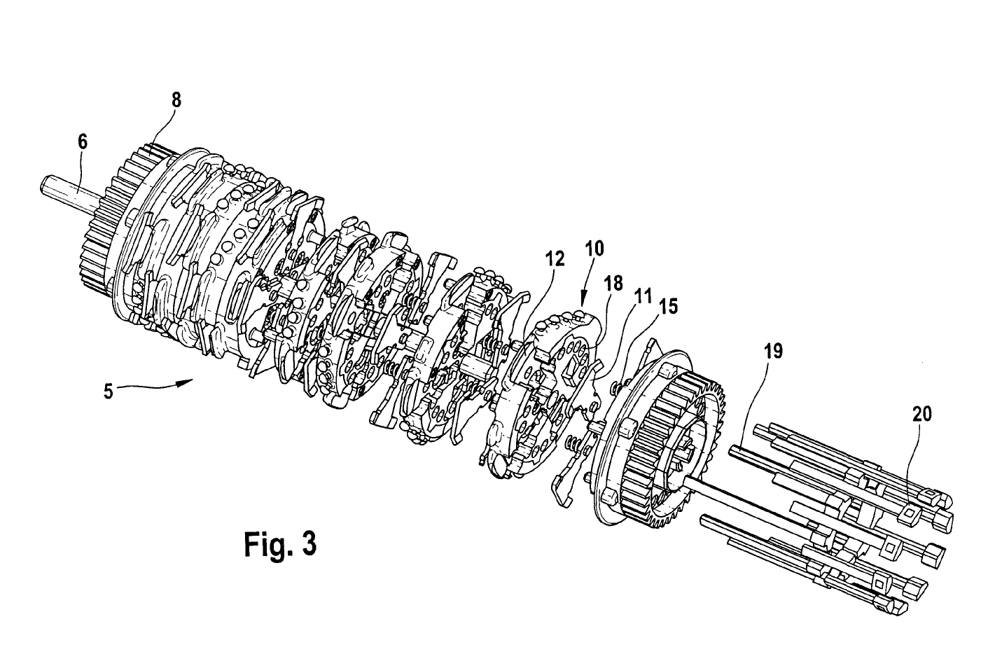

FIG. 2 shows a section through the epilation apparatus 1 in the region of the epilation head 3. A perspective exploded view of the rotatable cylinder 5 is shown in FIG. 3. The rotatable cylinder 5 is rotatably supported in the epilation head 3 by means of a shaft 6 such that the shaft 6 defines an axis of rotation 7 of the rotatable cylinder 5. One respective gear 8 is connected to the rotatable cylinder 5 in a rotationally rigid fashion in the region of the axial ends of the rotatable cylinder 5. One of the gears 8 is engaged with a driving pinion 9 that is driven by an electric motor (not shown) arranged in the interior of the housing 2 via a gear assembly that is also not illustrated in the figures. In the axial direction, the rotatable cylinder 5 is composed of a multitude of stacked components. The rotatable cylinder 5 features several carrier disks 10. Four movable clamping elements 11 are arranged on each carrier disk 10. In addition, four stationary clamping elements are provided per carrier disk 10, such that the stationary clamping elements are uniformly distributed over the circumference in the region of the outer radius of the carrier disk 10 and slightly offset relative to one another in the axial direction. The stationary clamping elements 12 are realized in one piece with the carrier disk 10 and preferably made of a plastic material. One movable clamping element 11 and one stationary clamping element 12 are respectively arranged axially adjacent to one another and collectively form a clamping device 13. The movable clamping elements 11 are respectively realized in the form of individual components and preferably made of a plastic material. The movable clamping elements 11 extend radially outward beginning at a radius that is larger than the radius of the shaft 6 of the rotatable cylinder 5. Due to their relatively small size in the radial direction, the movable clamping elements 11 have a relatively small mass and therefore can be easily set in motion and stopped again.

In the vicinity of their inner radial ends, the movable clamping elements 11 are supported such that they can be tilted relative to the axis of rotation 7 of the rotatable cylinder 5 and therefore moved toward and away from the respectively adjacent stationary clamping element 12. This makes it possible to open and close the clamping devices 13. Each movable clamping element 11 features a pin 14 that is oriented perpendicular to the principal surface of the movable clamping element 11 radially outside its bearing point, such that a spring element 15 in the form of a coil spring is threaded on the aforementioned pin. The pin 14 is sectionally realized in a barrel-shaped fashion in order to fix the spring element 15 on the pin 14. The spring element 15 is supported on the barrel-shaped region of the pin 14 with one end and on the base of a recess 16 in the carrier disk 10 with the other end. The pin 14 completely penetrates the movable clamping element 11 and extends into a depression 17 of the carrier disk 10 on the rear side of the movable clamping element 11, i.e., on the side that faces away from the spring element 15. The spring elements 15 prestress the movable clamping elements 11 such that they move away from the corresponding stationary clamping elements 12. Since separate spring elements 15 are provided, the prestress of each individual movable clamping element 11 is preserved in an unchanged fashion regardless of the condition of the other movable clamping elements 11.

The carrier disks 10 feature several axial through-bores 18 radially outside the recesses 16 or the depressions 17, respectively, such that the through-bores are arranged on a common reference circle and regionally aligned with one another in axially adjacent carrier disks 10. Rods 19 guided in the through-bores 18 are made of a plastic material and partly have a different length. One respective set of rods 19 extends through the through-bores 18 from each axial end of the rotatable cylinder 5 and ends no further than the axial center of the rotatable cylinder 5, such that each rod 19 ends at a different movable clamping element 11 and such that one rod 19 is respectively assigned to each movable clamping element 11. The rods 19 respectively feature a rod head 20 in the region of the axial ends of the rotatable cylinder 5, such that these rod heads are also made of a plastic material and widened with respect to the rod cross section. The rods 19 are preferably realized in one piece with the respectively assigned rod heads 20. The cross-sectional shape of the rod heads 20 is respectively realized such that rod heads 20 situated adjacent to one another in the circumferential direction are alternately extended radially inward and radially outward.

One respective lateral part 21 is arranged adjacent to the two axial end regions of the rotatable cylinder 5. The lateral parts 21 do not turn with the rotatable cylinder 5 and respectively feature a pressure element 22 with a control surface 23 on their side that faces the rotatable cylinder 5, namely in the region of the reference circle, on which the through-bores 18 are arranged. The pressure elements 22 are made of a plastic material. A bow spring 25 respectively engages into a groove 24 arranged in the side of the lateral parts 21 that faces away from the rotatable cylinder 5. The bow spring 25 axially presses the two lateral parts against the rotatable cylinder 5.

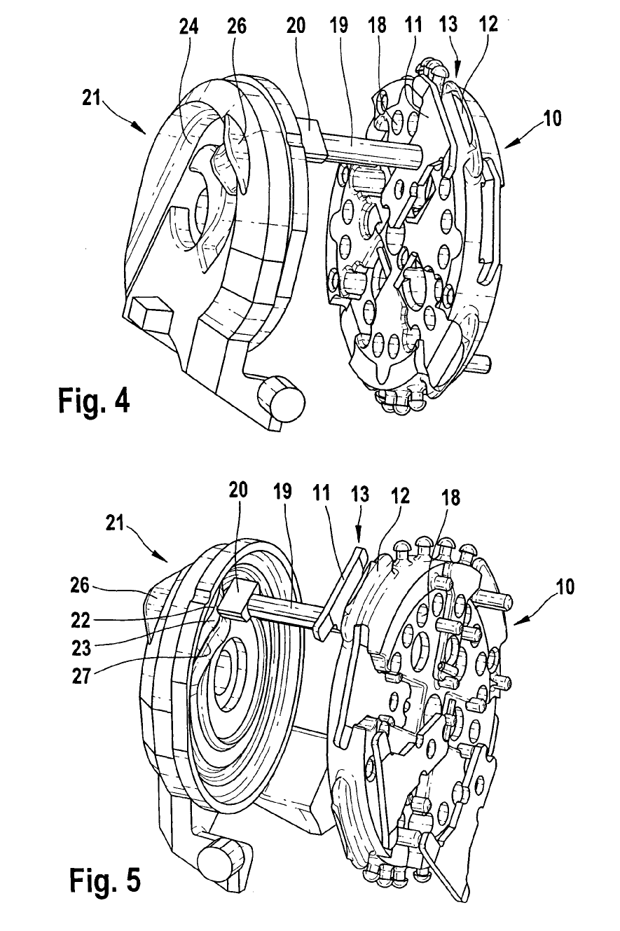

FIG. 4 shows an arrangement consisting of a lateral part 21 and a few components of the rotatable cylinder 5 in the form of a perspective representation. FIG. 5 shows the same arrangement in the form of a different perspective representation than FIG. 4. Only one rod 19 is respectively illustrated in FIGS. 4 and 5 so as to provide a better overview. A holding arrangement 26 for the bow spring 25 is arranged on the side of the lateral part 21 that faces away from the rotatable cylinder 5, namely in the region of the pressure element 22. The bow spring 25 is pressed against the lateral part 21 at this location. The pressure element 22 is shaped such that the control surface 23 forms an elevation that, referred to the circumferential direction of the lateral part 21, begins in the form of a steadily ascending ramp and ends in a steadily descending ramp. The pressure element 22 features a circumferentially extending groove 27 in its radial center with respect to the control surface 23, such that the groove serves as the lubricant depot. In FIGS. 4 and 5, the rotational position of the rotatable cylinder 5 relative to the lateral part 21 is chosen such that the rod 19 shown contacts the control surface 23 with its rod head 20 in the vicinity of the highest point of the control surface 23 of the pressure element 22. The clamping device 13, the movable clamping element 11 of which is contacted by the other end of the rod 19 shown, is almost completely closed.

In the operative state of the epilation apparatus 1, the gear 8 of the rotatable cylinder 5 is driven by the driving pinion 9 such that the rotatable cylinder 5 is set in rotation. Due to this rotational movement, the rods 19 successively slide over the control surface 23 of one of the pressure elements 22 with their heads 20. In this case, the bow spring 25 prevents the lateral parts 21 from giving way to the effect of the rods 19 to a significant degree and from yielding in the axial direction. Due to the contact with the control surface 23 of one of the pressure elements 22, the respective rods 19 are instead successively displaced toward the axial center of the rotatable cylinder 5. This means that the rods 19 arranged in the left half of the rotatable cylinder 5 are respectively displaced toward the right and the rods 19 arranged in the right half of the rotatable cylinder 5 are respectively displaced toward the left. The dynamics and the extent of these displacements depend on the shape of the control surfaces 23 of the pressure elements 22 that may also deviate from the embodiment shown. The movements of the rods 19 are transmitted to the movable clamping elements 11 that are respectively contacted by the rods 19. This causes the movable clamping elements 11 to move toward the respectively adjacent stationary clamping elements 12 against the restoring force of the spring elements 15 and to ultimately press against these stationary clamping elements such that the clamping devices 13 are successively closed. The closed state of the clamping devices 13 is respectively reached when the rod head 20 of the corresponding rod 19 contacts the highest point of the control surface 23 of one of the pressure elements 22. At this point in time, the respective clamping device 13 is arranged in a position in which it faces away from the housing 2 of the epilation apparatus 1 and is therefore situated in the vicinity of the principal surface. If a hair is located between the movable clamping element 11 and the stationary clamping elements 12 of this clamping device 13 shortly before this time, the hair is clamped between the clamping elements when the clamping device 13 is closed. Due to the rotational movement of the rotatable cylinder 5, the clamping device 13 continues its movement such that the clamped hair is plucked out of the skin.

The rotational movement of the rotatable cylinder 5 also causes the respective rod head 20 to continue its sliding motion on the control surface 23 of the pressure element 22 such that it moves away from the highest point of the control surface 23. Due to the restoring force of the spring element 15, the movable clamping element 11 is moved away from the adjacent stationary clamping element 12 by a distance that is defined by the shape of the control surface 23 of the pressure element 22 such that the clamping device 13 is opened. The plucked hair is released once the clamping device 13 is opened.

The opening movements of the clamping devices 13 as well as their closing movements are therefore defined by the shape of the control surfaces 23 of the pressure elements 22. The shape of the control surfaces 23 of the pressure elements 22 also defines the ranges of the angle of rotation, within which the clamping devices 13 are respectively opened and closed, as well as the pressing force that is respectively exerted upon the stationary clamping elements 12 by the movable clamping elements 11. In some embodiments, it is possible to realize the control surfaces 23 such that several clamping devices 13 are simultaneously held in the closed position by means of a control surface 23, such that the clamping forces generated by the clamping devices 13 can be identical. In addition, the control surfaces 23 may be designed such that the clamping devices 13 are respectively opened and closed several times per revolution of the rotatable cylinder 5.

In order to ensure that the hairs are reliably plucked out of the skin, the clamping devices 13 need to clamp the hairs so tightly that they do not slide out during the plucking process. This makes it necessary to respectively press the clamping elements 11 and 12 against one another with a sufficiently high pressure. This in turn means that a sufficiently high force needs to be exerted upon the movable clamping elements 11 by the rods 19. The contact areas between the rod heads 20 and the control surfaces 23 are subjected to significant stresses because the rods 19 need to slide over the control surface 23 of one of the pressure elements 22 with their rod heads 20 and are supported on this control surface 23. This is the reason why the disclosure proposes measures that make it possible to realize the pressure elements 22 as well as the rods 19 inclusive of the rod heads 20 in the form of plastic parts despite these high stresses.

One measure includes realizing the rod heads 20 with a larger cross section than the rods 19 such that the surface pressure is reduced. Another measure that can be taken alternatively or additionally includes supplying the contact regions between the rod heads 20 and the control surfaces 23 of the pressure elements 22 with a lubricant. The lubricant makes it possible to significantly reduce the friction between the rod heads 20 and the pressure elements 22 such that only little wear occurs. A lubricating film, for example, is formed in the contact regions between the rod heads 20 and the control surfaces 23 of the pressure elements 22, such that this lubricating film at least largely prevents the rod heads 20 from directly contacting the control surfaces 23 of the pressure elements 22 and such that a favorable wear behavior is achieved.

With respect to the lubricating measures, excessive amounts of lubricant should not be removed from the epilation apparatus 1, for example, when the apparatus is cleaned. This is the reason why, in some embodiments, it is preferred to use a solid lubricant that also withstands the effects of water and tensides to a certain degree. An adequate supply of lubricant to the contact regions between the rod heads 20 and the control surfaces 23 of the pressure elements 22 can be achieved, for example, by providing the lateral parts 21 with a lubricant filling. In the embodiment shown, part of the lubricating grease would be deposited in the groove 27 of the control surface 23 and therefore stored in the immediate vicinity of the contact region. In some embodiments, an adequate lubricating effect can also be achieved by realizing the control surfaces 23 of the pressure elements 22 and/or the surfaces of the rod heads 20 that come in contact with these control surfaces in an open-pored fashion such that the lubricant sufficiently adheres to these surfaces. A similar effect can also be achieved with the aid of recesses that are arranged in the rod heads 20 and/or in the control surfaces 23 of the pressure elements 22. In other embodiments, plastic materials with a lubricant incorporated therein for the manufacture of the rod heads 20 and/or the pressure elements 22 such that an external lubricant supply can be eliminated or provided as a supplement to the incorporated lubricant.

1. A motor-driven epilator comprising:
a housing;
a epilation head mounted on the housing, the epilation head comprising;
a rotatable cylinder rotatable about an axis of rotation and comprising:
clampind devices actuatable to take hold of and pluck hairs;
actuating elements operable to actuate the clamping devices; and
at least one pressure element with a control surface that is at least intermittently contacted by the actuating elements in a sliding fashion,
wherein the actuating elements as well as the pressure element are made of plastic.

2. The epilator according to claim 1, wherein one or more of the control surface of the pressure element and the regions of the actuating elements that contact the control surface are supplied with a lubricant.

3. The epilator according to claim 2, wherein the pressure element is arranged in a lateral part of the epilation head and the lubricant is stored in the lateral part.

4. The epilator according to claim 2, wherein the lubricant used consists of a solid lubricant.

5. The epilator according to claim 2, wherein one or more of the control surface of the pressure element and the regions of the actuating elements that contact the control surface have a lubricant adhesion enhancing surface structure.

6. The epilator according to claim 2, wherein one or more of the control surface of the pressure element and the regions of the actuating elements that contact the control surface have an open-pored surface.

7. The epilator according claim 2, wherein one or more of the control surfaace of the pressure element and the regions of the actuating elements that contact the control surface have a surface defining at least one recess.

8. The epilator according claim 2, wherein the control surface of the pressure element defines a circumferentially extending groove.

9. The epilator according claim 2, wherein one or more of the pressure element and the actuating elements are at least partly made of a plastic material with a lubricant incorporated therein.

10. The epilator according to claim 1, wherein the control surface of the pressure element comprises a ramp.

11. The epilator according claim 1, wherein a cross section fo the actuating elements is enlarged toward the region that contacts the control surface of the pressure element.

12. The epilator according claim 1, wherein the actuating elements are shaped.

13. The epilator accroding to claim 1, wherein the clamping devices each comprise a clamping element that is movable relative to the rotatable cylinder and a clamping element that is stationarily arranged on the rotatable cylinder.

14. The epilator according to claim 13, wherein the movable clamping elements comprise individual parts.

15. The epilator according to claim 13, wherein several stationay clamping elements are portions of one integral component.

16. The epilator according to claim 13, wherein the movable clamping elements are made of metal.

17. The epilator according to claim 13, wherein the stationary clamping elements are made of plastic.

18. The epilator according to claim 1, further comprising spring elements operable to open the clamping devices.

19. An epilation head for an epilator the epilation head comprising:
a rotatable cylinder, which rotates about an axis of rotation, the cylinder comprising:
clamping devices operable to take hold and pluck hairs;
actuating elements operable to actuate the clamping devices; and
at least one pressure element with a control surface that is at least intermittently contacted by the actuating elements in a sliding fashion,
wherein the actuating elements as well as the pressure element are made of plastic.
